(19) United States
(12) Patent Application Publication
(10) Pub. No.: US 2024/0366360 A9
(43) Pub. Date: Nov. 7, 2024

(54) SELF-EXPANDING DEVICES AND RELATED PROCESSES

(71) Applicant: Sonorous NV, Lake Forest, CA (US)

(72) Inventors: Randall Takahashi, Mission Viejo, CA (US); Dawson Le, Garden Grove, CA (US); Dave Ferrera, Coto de Caza, CA (US)

(21) Appl. No.: 17/584,887

(22) Filed: Jan. 26, 2022

Related U.S. Application Data
(60) Provisional application No. 63/142,786, filed on Jan. 28, 2021.

(51) Int. Cl. A61F 2/06

(52) U.S. Cl. CPC A61F 2/06; A61F 2002/0081; A61F 2002/068; A61F 2210/0076

(57) ABSTRACT

New devices use baffles and multiple diameters to treat and prevent ringing in the ears by altering pressure gradients in subject sinuses/vessels. Novel braided stents with variable radial force based upon wires added and woven allows 7-11 cm therapies, for example, to resolve pulsatile tinnitus in patients under conscious sedation.

### CROSS-REFERENCE TO RELATED APPLICATION

This application claims the full Paris Convention benefit of, and priority to, U.S. provisional patent application Ser. No. 63/142,786, filed Jan. 28, 2021. This substitute specification is being lodged into the file wrapper to correct a formality regarding line spacing in the claims. This application contains no new matter.

### OBJECTS AND SUMMARY OF THE INVENTIONS

The field of the invention is compositions, devices, and methods of use and manufacturing of devices, both conceptually and in terms of structure (specifically focused upon their function) for changing pressure of blood flow within a subject blood vessel. Those skilled in the art understand this definitional approach to embrace any related art used during procedures having, for example, a change in blood flow, wherein symptoms of previously bothersome tinnitus are mitigated. The present inventions comprise novel enhanced mechanical self-expanding devices having at least about two or more diameters. Modulating and baffling blood flow is achieved via improved placement of different diameter stenting and capture means as shown in the figure in schematic cartooned form, along with known and innovative coverings and coatings for the subject devices. Also provided are materials and properties impacting flow dynamics linked to the solutions claimed herein and embraced within the scope of the instant application for United States Letters Patent, as herewith first disclosed in any form or reduced to practice by this written description at least as early the week of Jan. 28, 2021.

### BRIEF DESCRIPTION OF THE DRAWINGS

Various preferred embodiments are described herein with references to the drawings in which merely illustrative views are offered for consideration, whereby:

FIG. 1 shows a schematic barrel stent means for countering the dynamics of water-hammer effected blood flow, among other things, schematic or cartooned to show an infrastructure which is variously shaped, styled, covered and coated (as with ePTFE/ptfe/PU/etc.) to achieve and disclose in a broad sense the genus of the present inventions by full denouement of this species.

FIG. 2 schematically shows a stent flow diverter to resolve tinnitus.

FIG. 3 schematically shows a stent flow diverter to resolve tinnitus.

FIG. 4 shows the process of Sonorous NV treatment for tinnitus by using the instant stents or flow diverters.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION OF THE INVENTIONS

Expressly incorporated by reference as if fully set forth herein in its entirety is US2021/0205105 (U.S. Ser. No. 17/137,324) which is assigned to instant Assignee. Likewise, U.S. Pat. No. 9,585,741 is fully incorporated herein by reference, along with U.S. Ser. No. 14/082,019, filed Nov. 15, 2013, along with their respective reference patents and publications.

The description includes information that may be useful in understanding the present invention. It is not an admission that any of the information provided herein is prior art or relevant to the presently claimed invention, or that any publication specifically or implicitly referenced is prior art.

As claimed below, solutions to the problem of tinnitus are provided. Avoiding tinnitus is among the objects of the instant teachings, it is respectfully submitted.

In most cases, changing the pressure via a blood flow diverter results in relief from tinnitus, in both pulsatile and non-pulsatile forms.

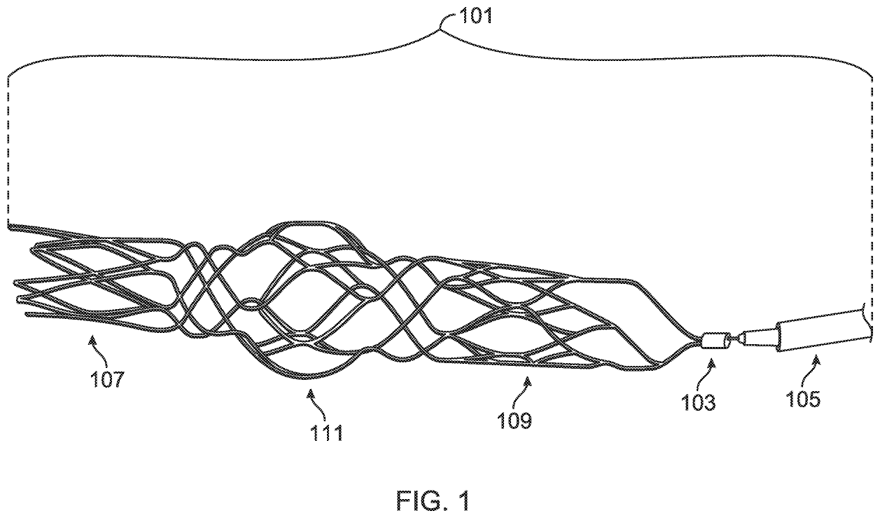

Referring now to FIG. 1, and to US2021/0205105, exemplary stenting member or any related types of known and later developed device(s) 101 is comprised of materials which artisans understand render the same self-expanding (Nitinol® alloys, stainless steel, and other materials which have memory), is delivered via sheath 105, and detachable at juncture 103.

Artisans understand that said device's main body 109/111/107 has more than one, often but not preferably more than two, respective diameters. Likewise, at least one of the larger baffle 111, and less large baffles 107/109 can be covered with materials like polyurethanes, PTFE, and the like, to change blood flow dynamics.

By way of further exemplary embodiments, the present inventive subject matter is drawn to compositions, devices, and methods of pressure modulating devices that have a mechanical self expanding device with two or more than two diameters, in this embodiment only. Referring now to FIG. 1, one is sized appropriately for the targeted occluded vessel of 3-6 mm, and a second diameter proximal to the main diameter that is sized for vessel diameters of the internal carotid, common carotid Basilar or vertebral arteries of 9-10 mm. Having a larger proximal diameter will act to baffle the rate of blood flow.

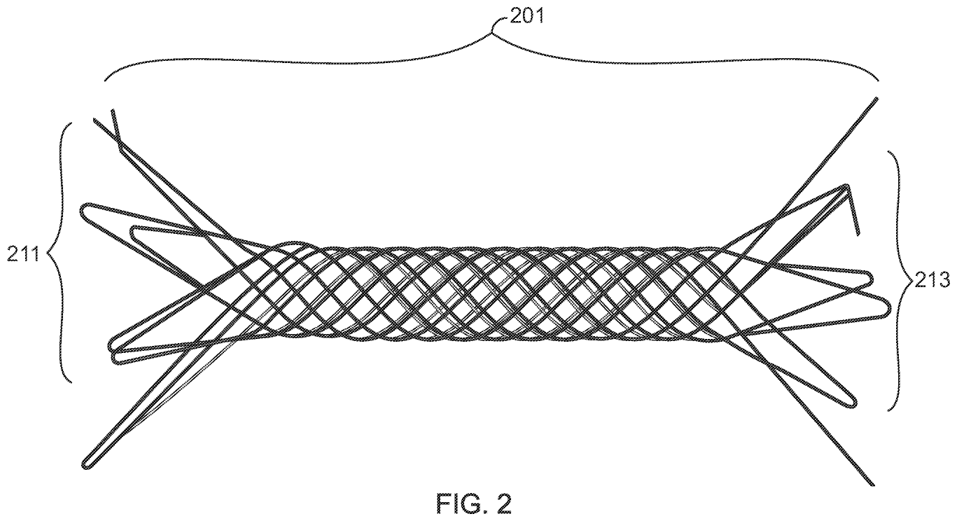

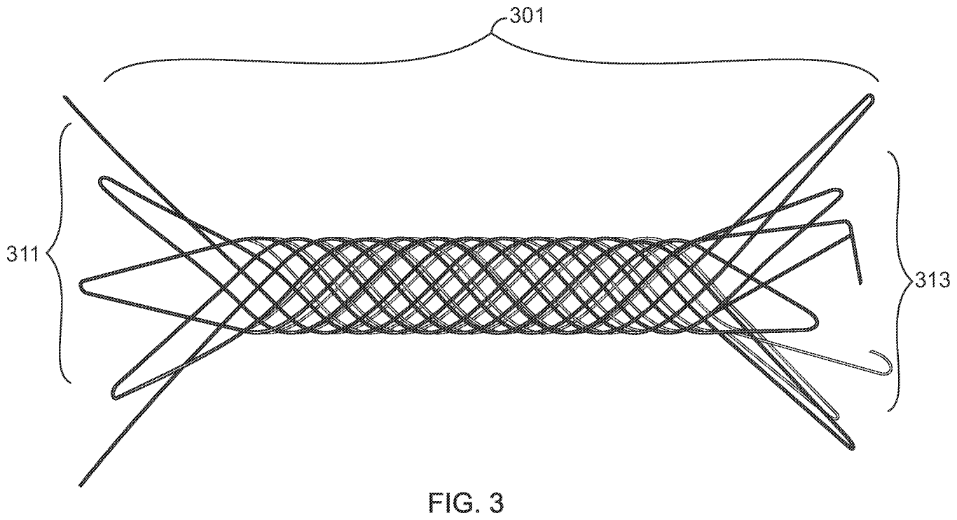

FIGS. 2 and 3 offer for consideration a system effective for altering venous flow characteristics which comprises a pressure gradient changer delivered to the bifurcation of the superior sagittal sinus. Namely, the transverse sinus between the origin of the straight sinus on either right or left branches. In addition, the larger baffle can be covered with a material such as PTFE or polyurethane film or a braid of polymer or metal to further enhance the baffle properties of the larger diameter.

The illustrative “barrel stent” is effective in several forms. The bulge can be more proximal and adjacent to the guidewire junction.

Referring now to FIGS. 2 and 3, a hand-woven braided stent provided the most reliable treatment for tinnitus, pulsatile or otherwise. Stent 201 changes the pressure gradient, resolving said issues. Crowns 211/213 allow for optimized placement, delivery and lodgment—either temporarily or permanently. FIG. 3 shows a multi-wire version 301 having higher radial force and larger size. Likewise, crowns 311/313 provide optimized delivery. Also the distal tip can be configured with closed end or porous end. The distal tip can be closed with a laser cut configuration and/or covered with PTFE, or a braid of polymer or metal.

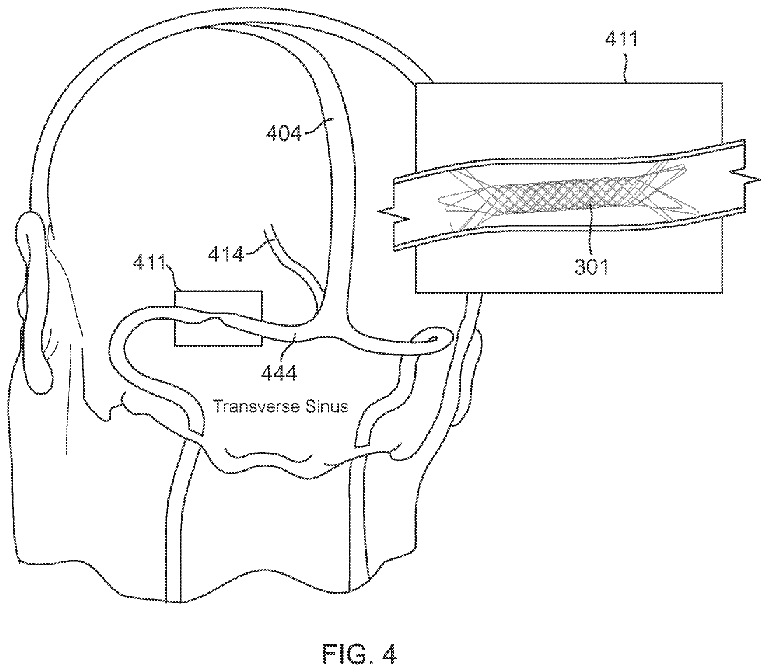

Referring now to FIG. 4, revolutionary tinnitus-resolving paradigms are fully set forth, in the exemplary and illustrative, but not limiting, figure offered for consideration. Artisans understand any A-V malformations, stenoses, or other venous bulb issues are all addressed, the same as exemplary stent 301 (shown in detail as 411) within the transverse sinus 444. Any related site within superior sagittal sinus 404, straight sinus 414, or other vessels is addressed this way. Conscious sedation allows for instantaneous resolution notation, with sensor (not shown) from U.S. patent publication US2021/0205105.

Various objects, features, aspects, and advantages of the inventive subject matter will become more apparent from the following detailed description of preferred embodiments, along with the accompanying drawing figure.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary, and that the actual parameters, dimensions, materials, and/or configurations will depend upon the specific application or applications for which the teachings of the present disclosure is/are used.

Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment,” means that a particular feature, structure, or characteristic described in connection with the embodiment is included in at least one embodiment. Thus, appearances of the phrases “in one embodiment”, or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention, and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A process for treating at least one of tinnitus and sinus-based stenosis, which comprises, in combination, delivery of a system effective for altering venous pressure and flow characteristics.

2. The process of claim 1, further comprising venous stenting with endovascular pressure feedback.

3. The process of claim 2, treating at least one of pulsatile or non-pulsatile tinnitus.

4. The process of claim 3, the etiology being vascular and classified based upon location as venous, arterial, or A-V.

5. The process of claim 4, said venous stent/flow diverter providing for symptomatic control and resolution of tinnitus.

6. The process of claim 5, whereby post stent/flow diverter deployment, no pressure gradient exists further in addressed sinus, vein, or artery.

7. The process of claim 6, further comprising conscious sedation and general anesthesia.

8. The process of claim 7, driven by a stent/flow diverter with more than four crowns.

9. The process of claim 7, said stent/flow diverter having at least about 5-8 crowns.

10. A novel enhanced system, which among other things, comprises blood flow baffling-means, the structure including in combination:
at least a stent/flow diverter capable of self-expansion, having more than one diameter.

11. The system of claim 10, wherein an at least two diameter version is used, particularly in a neurovascular intervention.

12. The system of claim 11, further comprising at least one of said diameters being sized for the target occluded vessel, and the second being a diameter close to the proximal main diameter that is sized for another secondary vessel.

13. The system of claim 12, wherein the secondary vessel is at least one of the internal carotid, common carotid, Basilar and vertebral arteries.

14. The system of claim 12, further comprised of covering, coating, surface, surface treatment or outer layer effect whereby the flow dynamics of blood are impacted.

15. The stenting-member having at least one barrel stent section or a zone of different diameter of claim 12, and whereby the barrel stent section is more proximal to and adjacent to the guide wire junction and vice-versa with any number of sections and coverings to achieve desired blood baffling.

16. The system of claim 15, whereby a distal tip may be configured with closed or porous ends;
a distal tip closed with a laser cut (or woven/braided) configuration; and covered or coated with PTFE or polymer or alloy or metal or surface topography or biologics and any other agents.

17. The process of claim 9, whereby conscious sedation enables a patient to perceive the resolution of the tinnitus.

18. Products by the process of claim 17, being out-patient procedures to resolve tinnitus.

19. Products by the process of claim 18, further comprising data regarding pressure gradient changes, best devices for same.

20. Data as a product of the process of claim 19, dedicated to a patient-blind registry.
